(19) United States
(12) Patent Application Publication
(10) Pub. No.: US 2005/0010995 A1
(43) Pub. Date: Jan. 20, 2005

(54) Connecting structure for a frame and a head strap of a pair of swimming/diving goggles

(76) Inventor: Terry Chou, Tainan City (TW)

Correspondence Address: CHARLES E. BAXLEY, ESQ., 90 JOHN STREET, THIRD FLOOR, NEW YORK, NY, 10038, US

(21) Appl. No.: 10/621,151

(22) Filed: Jul. 15, 2003

(51) Int. Cl. A61F 9/02

(52) U.S. Cl. 2/428

(57) ABSTRACT

A pair of swimming/diving goggles includes a soft frame having two outer sides each having an engaging portion, two lenses mounted in the soft frame, a head strap, and two connecting blocks respectively mounted to the engaging portions of the soft frame. Each connecting block is pull-resistant and includes a through-hole for engaging with the head strap. Each connecting block is at least partially embedded in and thus fixed by a plastic material for forming the soft frame. The soft frame is bendable according to a head contour of a user, providing an intimate contact with two sides of the head of the user. The respective connecting block is not deformed when the head strap is pulled.

### BACKGROUND OF THE INVENTION

1. Field of the Invention

The present invention relates to a connecting structure for a frame and a head strap of a pair of swimming/diving goggles. In particular, the present invention relates to a soft frame of a pair of swimming/diving goggles providing improved comfortable contact with the user's head, improved pull-resistance, and an aesthetically pleasing appearance.

2. Description of the Related Art

FIGS. 8 and 9 of the drawings illustrate a pair of conventional swimming/diving goggles including two rigid lenses 1′, two padding members 3′, and a head strap 2′. Each lens 1′ has a peripheral flanged portion 11′ on which an engaging portion 12′ is formed for engaging which an associated end of the head strap 2′. Each padding member 3′ is mounted to an inner side of an associated lens 1′. Each padding member 3′ is in intimate contact with an associated eye's socket of the user under the action of the clamping force of the head strap 2′. However, the peripheral flanged portion 11′ of the respective lens 1′ and the head strap 2′ are rigid and thus may cause injury to the user when impinged. Further, the peripheral flanged portion 11′ of the respective lens 1′ and the head strap 2′ provide poor comfortable contact with the user's head and poor feeling to touch.

FIGS. 10 and 11 illustrate another pair of conventional swimming/diving goggles including two rigid lenses 4′, a soft frame 5′, and a head strap 6′. The lenses 4′ are mounted to a front portion of the frame 5′. The frame 5′ has two sides 51′ each having an engaging portion 52′ for engaging with an associated end of the head strap 6′, with each engaging portion 52′ having a through-hole 521′ through which an associated end of the head strap 6′ extends. The sides 51′ of the soft frame 5′ provide improved comfortable contact with the head contour of the user than the pair of swimming/diving goggles of FIGS. 8 and 9. However, the pull-resistance is sacrificed. More specifically, as illustrated in FIG. 11, the engaging portions 52′ are apt to be largely deformed when subject to the force exerted to the engaging portions 52′ while pulling the head strap 6′. The distal portions 522′ of the engaging portions 52′ might even be broken.

FIG. 12 illustrates a portion of a further pair of conventional swimming/diving goggles including two rigid lenses 7′, a soft frame 8′, a head strap 9′ and two connecting blocks 91′. Formed on each of two sides of the frame 8′ is a connecting portion 81′ that has a through-hole 811′. Each connecting block 91′ is made of rigid material and has a width greater than that of an associated through-hole 811′. When the respective connecting block 91′ is pulled by the head strap 9′, the respective connecting block 91′ would not deform largely, preventing a distal portion 812′ of the respective engaging portion 81′ from being broken. However, the respective rigid connecting block 91′ is located outside the respective through-hole 811′ and thus fails to provide comfortable contact with the user's head. Further, the respective connecting block 91′ is not fixed before engaging with the head strap 9′ such that the respective connecting block 91′ is moved to a position (see the phantom lines shown in FIG. 12) away from the respective through-hole 811′, failing to provide an aesthetically pleasing appearance.

FIGS. 13 and 14 illustrate still another pair of conventional swimming/diving goggles including a frame 92′, two lenses 94′, and a padding member 95′. The frame 92′ includes a rigid inner frame 921′ embedded in a soft outer frame 922′. Each end of the head strap 93′ is engaged with an engaging portion 923′ on an associated side of the frame 92′. Thus, no deformation occurs in the rigid inner frame 921′ when the head strap 93′ is pulled. However, the rigid inner frame 921′ could not deform after formation, i.e., the rigid inner frame 921′ could not bend according to the head contour of the user, generating a gap “a” (FIG. 14) between the frame 92′ and the head of the user and failing to provide an intimate contact with the user's head.

### SUMMARY OF THE INVENTION

An object of the present invention is to provide a pair of swimming/diving goggles capable of providing improved comfortable contact with the user's head, improved pull-resistance, and an aesthetically pleasing appearance.

A pair of swimming/diving goggles in accordance with the present invention includes a soft frame having two outer sides each having an engaging portion, two lenses mounted in the soft frame, a head strap, and two connecting blocks respectively mounted to the engaging portions of the soft frame. Each connecting block is pull-resistant and includes a through-hole for engaging with the head strap. Each connecting block is at least partially embedded in and thus fixed by a plastic material for forming the soft frame.

In an embodiment of the invention, the respective connecting block is a ring or substantially C-shaped. The respective engaging portion of the soft frame is a ring at least partially embedded in the respective engaging portion of the soft frame.

The soft frame is bendable according to a head contour of a user, providing an intimate contact with two sides of the head of the user. The respective connecting block is not deformed when the head strap is pulled.

Other objects, advantages, and novel features of the invention will become more apparent from the following detailed description when taken in conjunction with the accompanying drawings.

### BRIEF DESCRIPTION OF THE DRAWINGS

FIG. 1 is a perspective view of a portion of an embodiment of a pair of swimming/diving goggles in accordance with the present invention.

FIG. 2 is a top view, partly sectioned, of the portion of the pair of swimming/diving goggles in FIG. 1.

FIG. 3 is a sectional view similar to FIG. 2, illustrating pulling of the head strap of the pair of the swimming/diving goggles.

FIG. 4 is a sectional view similar to FIG. 3, illustrating a modified embodiment of the pair of swimming/diving goggles in accordance with the present invention.

FIG. 5 is a perspective view of a portion of another modified embodiment of the pair of swimming/diving goggles in accordance with the present invention.

FIG. 6 is a top view, partly sectioned, of a portion of a further modified embodiment of the pair of swimming/diving goggles in accordance with the present invention.

FIG. 7 is a perspective view of a portion of still another modified embodiment of the pair of swimming/diving goggles in accordance with the present invention.

FIG. 8 is a perspective view, partly exploded, of a pair of conventional swimming/diving goggles.

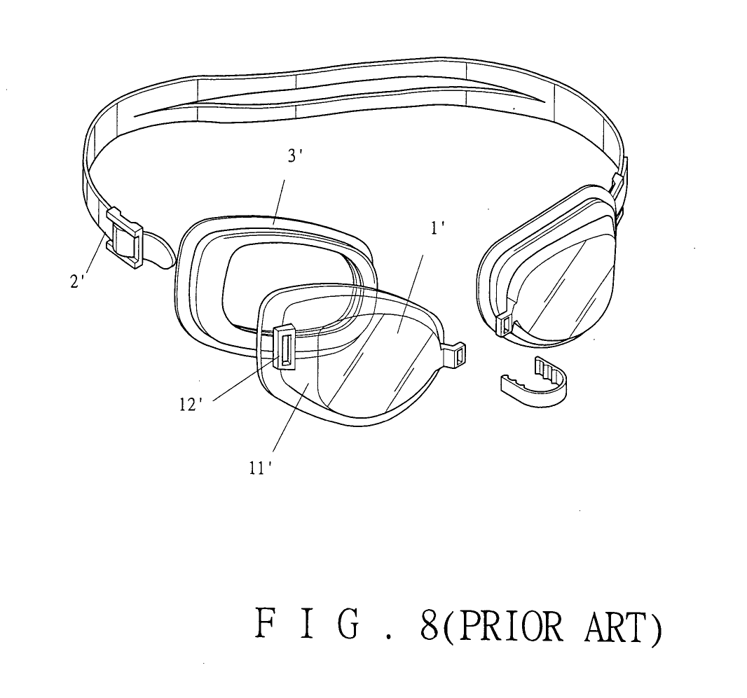

FIG. 9 is a top view, partly sectioned, of a portion of the pair of conventional swimming/diving goggles in FIG. 8.

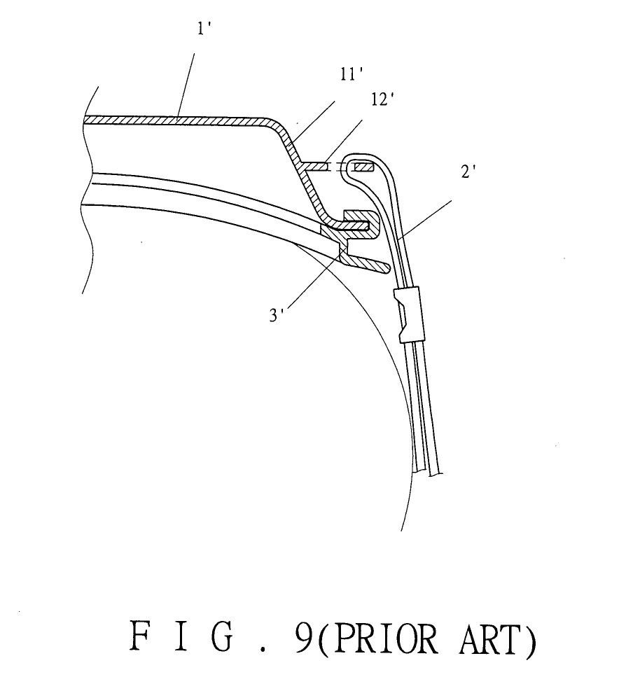

FIG. 10 is a perspective view of another pair of conventional swimming/diving goggles.

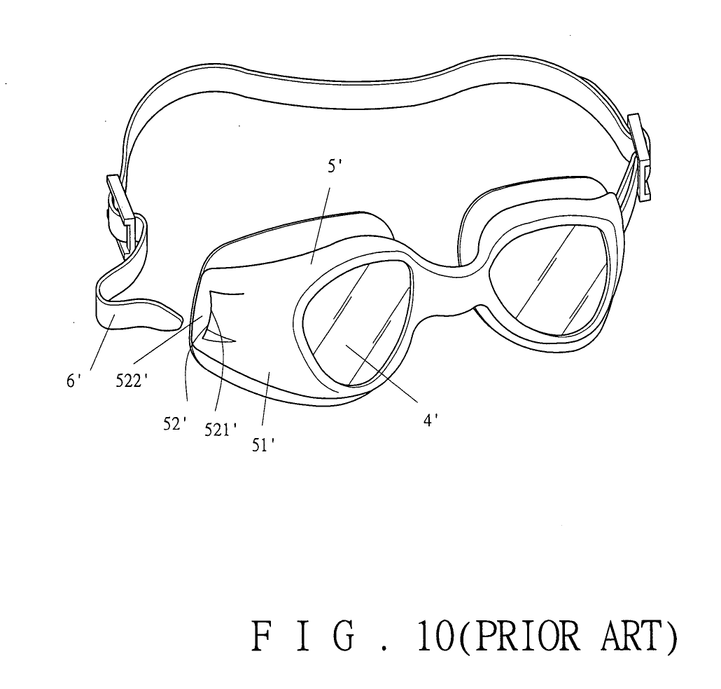

FIG. 11 is a top view, partly sectioned, of a portion of the pair of conventional swimming/diving goggles in FIG. 10.

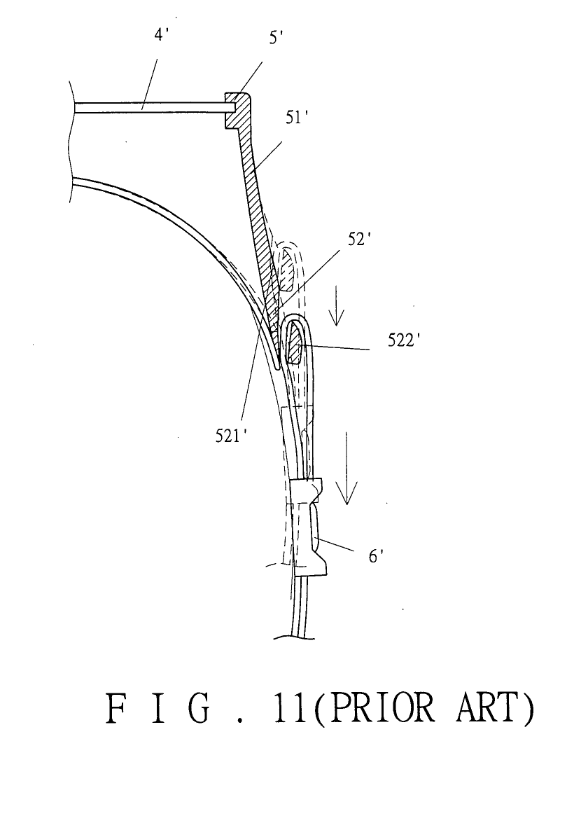

FIG. 12 is a top view, partly sectioned, of a portion of a further pair of conventional swimming/diving goggles.

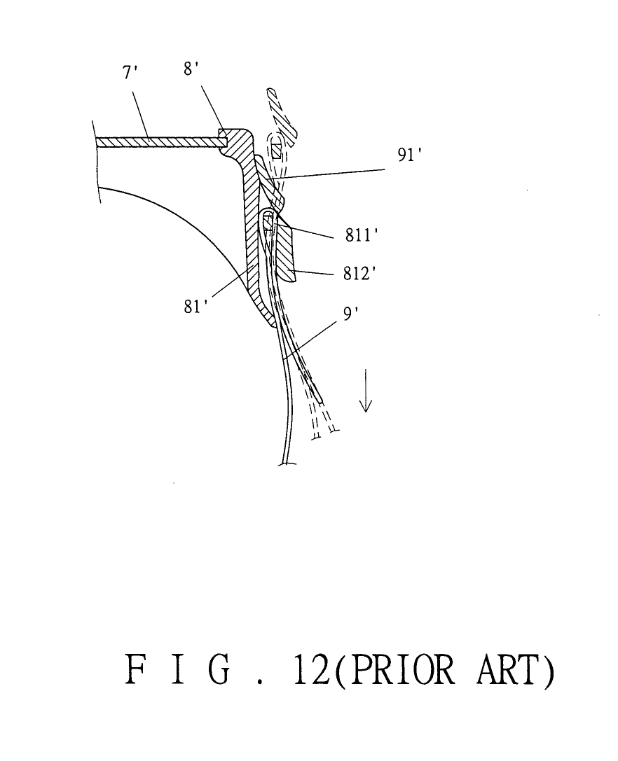

FIG. 13 is an exploded perspective view of still another pair of conventional swimming/diving goggles.

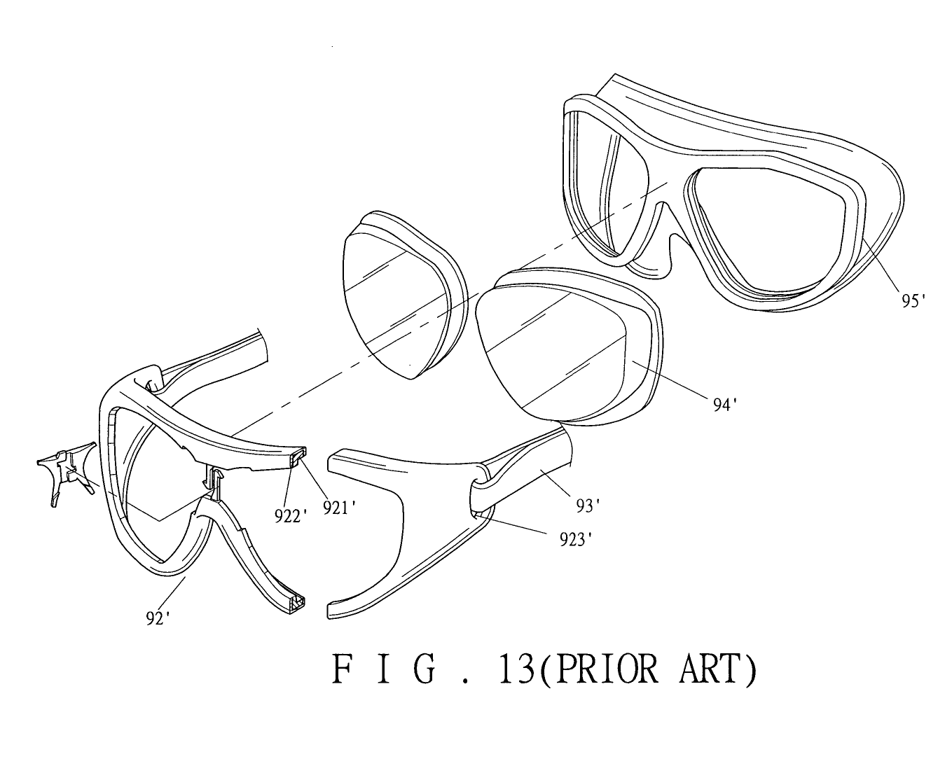

FIG. 14 is a top view, partly sectioned, of a portion of the pair of conventional swimming/diving goggles in FIG. 13.

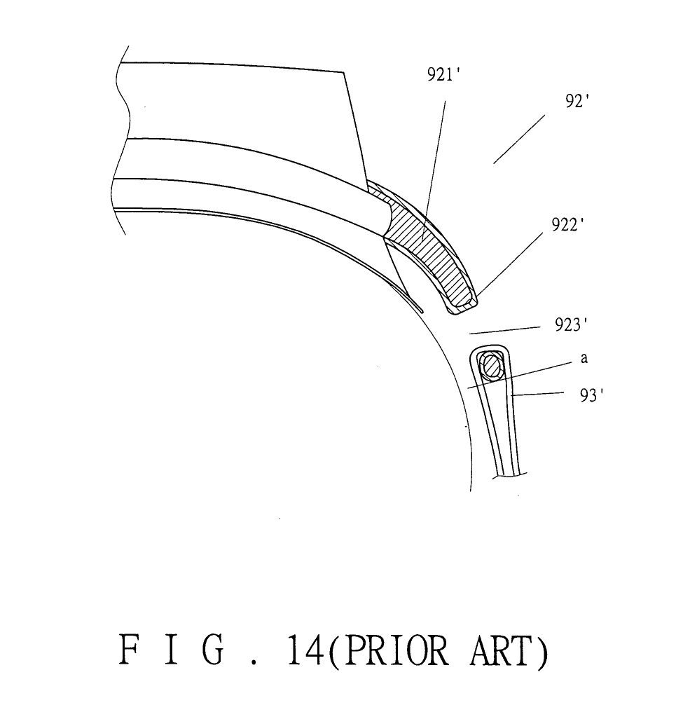

### DETAILED DESCRIPTION OF THE PREFERRED EMBODIMENT

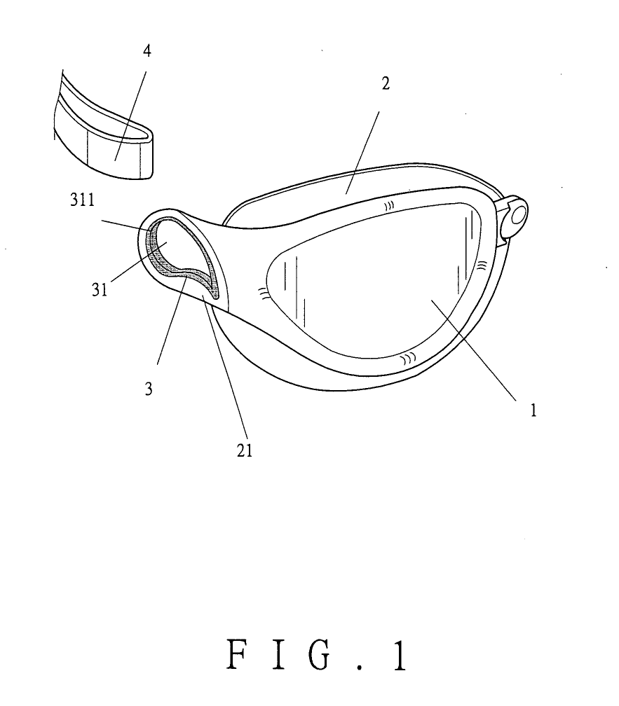

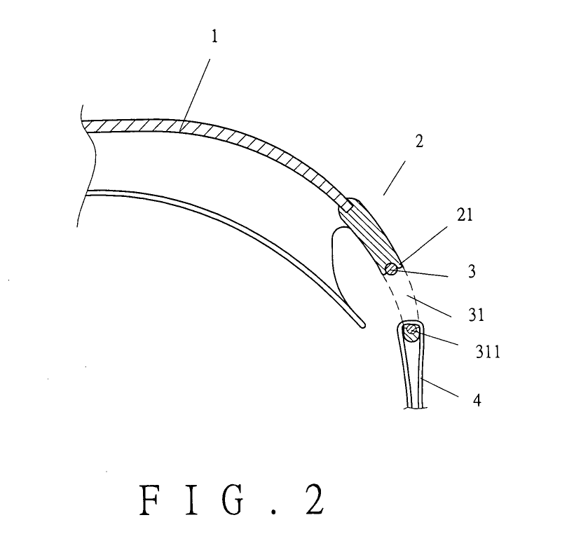

Referring to FIGS. 1 and 2, an embodiment of a pair of swimming/diving goggles in accordance with the present invention generally comprises two lenses 1 (only one is shown), a soft frame 2, two connecting blocks 3, and a head strap 4. The lenses 1 are mounted in a front portion of the soft frame 2. The soft frame 2 is flexible and bendable and has an engaging portion 21 on each of two outer sides thereof for engaging with the head strap 4.

Each connecting block 3 is pull-resistant (preferably made of rigid material) and includes a through-hole 31. In this embodiment, the respective engaging portion 21 of the soft frame 2 is in the form of a ring, and the respective connecting block 3 is in the form of a ring for engaging with the head strap 4. The respective connecting block 3 is preformed and then placed in a mold (not shown) and in contact with an inner periphery of the respective ring-like engaging portion 21 of the frame 2 that is also placed in the mold. Next, the respective connecting block 3 is partially enclosed and thus fixed by the plastic material for forming the respective ring-like engaging portion 21. Thus, the respective connecting block 3 is partially embedded in the respective ring-like engaging portion 21.

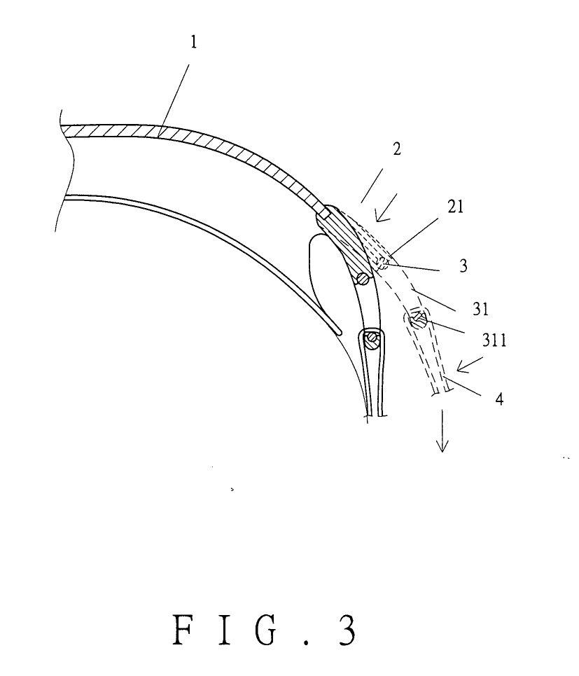

As illustrated in FIG. 3, the head strap 4 is extended through the through-hole 31 and wound around the respective connecting block 3. When in use, the soft frame 2 may bend according to the head contour of the user, providing a comfortable contact with two sides of the head of the user. Further, the respective connecting block 3 is so pull-resistant that an end portion 311 of the respective connecting block 3 would not deform or break when the head strap 4 is pulled. Further, the respective connecting block 3 is embedded in the frame 2, preventing the respective connecting block 3 from disengaging from the frame 2 and providing an aesthetically pleasing effect, as the respective connecting block 3 and the frame 2 are integrally formed as a one-piece member.

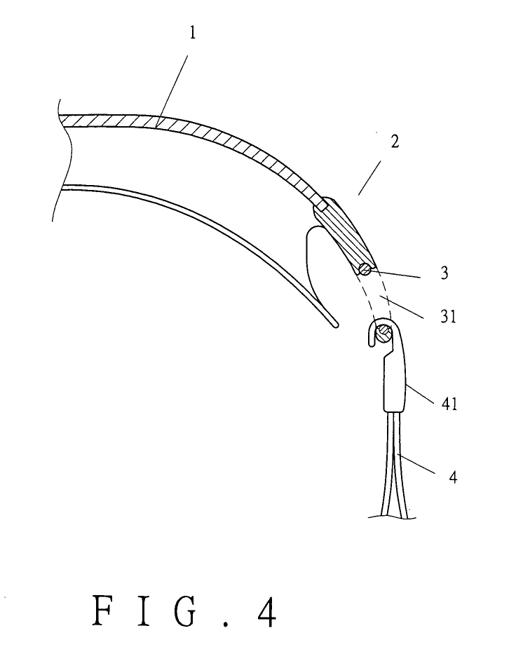

FIG. 4 illustrates a modified embodiment of the invention, wherein the head strap 4 includes a connection block 41 mounted thereto for engaging with the through-hole 31 of the respective connecting block.

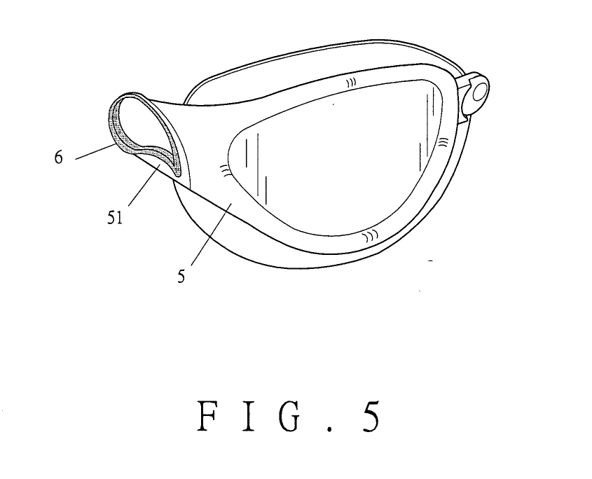

FIG. 5 illustrates another modified embodiment of the invention, wherein the respective engaging portion (now designated by 51) of the frame (now designated by 5) is engaged with an upper portion of the respective connecting block (now designated by 6), providing a reliable engaging effect.

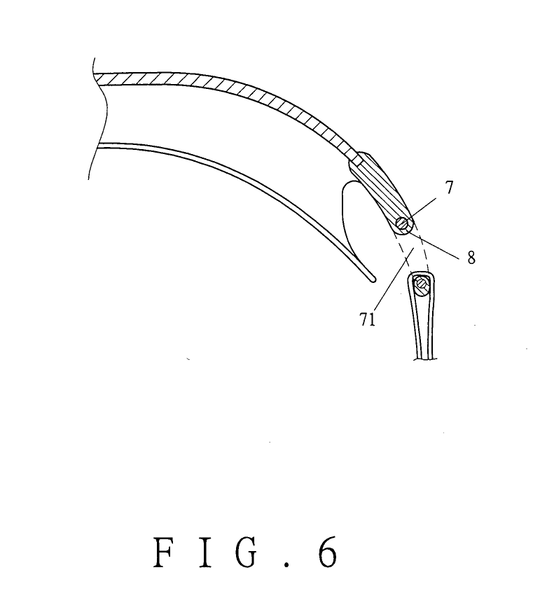

FIG. 6 illustrates a further modified embodiment of the invention, wherein the respective connecting block (now designated by 7) having a through-hole (now designated by 71) is completely enclosed by the plastic material for forming the soft frame (now designated by 8), providing improved comfort feeling to touch and improved engaging effect.

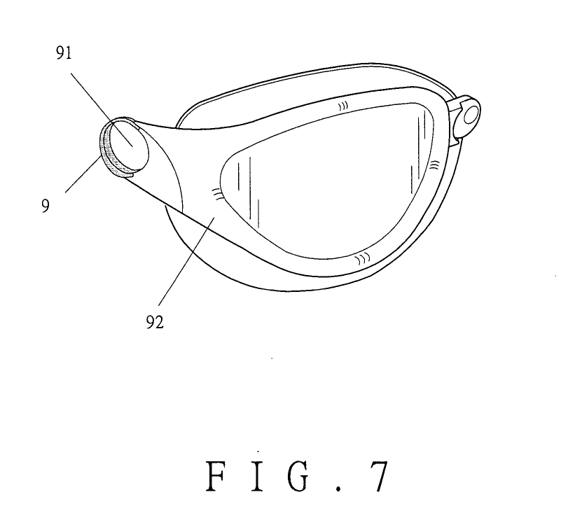

FIG. 7 illustrates still another modified embodiment of the invention, wherein the respective connecting block (now designated by 9) is substantially C-shaped (see open portion 91). The respective connecting block 9 is integrally formed with the soft frame (now designated by 92), with the open portion 91 of the respective connecting block 9 being enclosed by the plastic material for forming the soft frame 92, providing a reliable engaging effect.

Although the invention has been explained in relation to its preferred embodiment, it is to be understood that many other possible modifications and variations can be made without departing from the scope of the invention as hereinafter claimed.

1. A pair of swimming/diving goggles comprising:
a soft frame having two outer sides each having an engaging portion;
two lenses mounted in the soft frame;
a head strap; and
two connecting blocks respectively mounted to the engaging portions of the soft frame, each said connecting block being pull-resistant and including a through-hole for engaging with the head strap, each said connecting block being at least partially embedded in and thus fixed by a plastic material for forming the soft frame.

2. The pair of swimming/diving goggles as claimed in claim 1, wherein the head strap includes a connection block attached thereto for engaging the respective connecting block.

3. The pair of swimming/diving goggles as claimed in claim 1, wherein the respective connecting block is a ring.

4. The pair of swimming/diving goggles as claimed in claim 1, wherein the respective connecting block is substantially C-shaped.

5. The pair of swimming/diving goggles as claimed in claim 1, wherein the respective engaging portion of the soft frame is a ring.

6. The pair of swimming/diving goggles as claimed in claim 5, wherein the respective connecting block is at least partially embedded in the respective engaging portion of the soft frame.

7. The pair of swimming/diving goggles as claimed in claim 1, wherein the soft frame is bendable according to a head contour of a user, providing an intimate contact with two sides of the head of the user, and wherein the respective connecting block is not deformed when the head strap is pulled.

8. The pair of swimming/diving goggles as claimed in claim 1, wherein the soft frame and the connecting blocks are integrally formed as a one-piece member.
